(19) United States
(12) Patent Application Publication
(10) Pub. No.: US 2025/0169830 A1
(43) Pub. Date: May 29, 2025

(54) DRILL GUIDES FOR FACILITATING PLACEMENT OF SURGICAL STAPLES INTO PRE-DRILLED HOLES AND RELATED METHODS OF USING THE SAME

(71) Applicant: INTELIVATION TECHNOLOGIES LLC, Saint Simons Island, GA (US)

(72) Inventors: David Murphy, Saint Simons Island, GA (US); Amit Sinha, Saint Simons Island, GA (US); Tyler Gleason, Saint Simons Island, GA (US)

(21) Appl. No.: 18/837,304

(22) PCT Filed: Feb. 9, 2023
(86) PCT No.: PCT/US2023/012690

§ 371 Date: Aug. 9, 2024

Related U.S. Application Data
(60) Provisional application No. 63/308,457, filed on Feb. 9, 2022.

(51) Int. Cl. A61B 17/17; A61B 17/064; A61B 17/068

(52) U.S. Cl. CPC A61B 17/17; A61B 17/0682; A61B 2017/0645

(57) ABSTRACT

A drill guide for facilitating placement of a surgical staple into pre-drilled holes formed in a patient may include a support structure and a plurality of guide members coupled to the support structure. Each of the guide members may have a proximal end and a distal end disposed opposite one another along a longitudinal axis of the guide member. Each of the guide members may include a tubular portion extending from the proximal end toward the distal end of the guide member and a track portion extending from the tubular portion toward the distal end of the guide member. The tubular portion may define a through passage extending along the longitudinal axis of the guide member. The track portion may define an open channel in communication with the through passage and extending along the longitudinal axis of the guide member.

### CROSS-REFERENCE TO RELATED APPLICATIONS

This application claims the benefit of U.S. Provisional Application No. 63/308,457, filed Feb. 9, 2022, which is incorporated by reference in its entirety.

### FIELD OF THE DISCLOSURE

The present disclosure relates generally to drill guides and more particularly to drill guides for facilitating placement of surgical staples into pre-drilled holes and related methods of using the same.

### BACKGROUND OF THE DISCLOSURE

Various types of drill guides may be used in orthopedic surgery to facilitate drilling of holes at desired locations in bone structures of a patient. Such drill guides generally may be used to ensure that locations and trajectories of the drilled holes correspond to requirements of a particular surgical procedure being performed. According to certain procedures, a drill guide may be used for drilling a pair of holes and then removed from the surgical site to allow for surgical hardware, such as a staple, to be advanced into the pre-drilled holes.

Certain existing drill guides may include a pair of guide members each formed as a tubular structure having a through passage extending along a longitudinal axis of the guide member from a proximal end to a distal end of the guide member. The guide members may be mounted to a support structure of the drill guide, with the longitudinal axes of the guide members having a desired orientation relative to one another, such as parallel to one another. During use of the drill guide, the guide members may be positioned at a desired location relative to a surgical site of a patient, and then a drill may be advanced through the through passages of the guide members, one at a time, to form respective holes in bone structures of the patient. After drilling the holes, the drill guide may be removed from the surgical site, and surgical hardware may be advanced into the pre-drilled holes. For example, a surgical staple may be used to facilitate fixation of the bone structures, with one arm of the staple being advanced into one of the pre-drilled holes and the other arm of the staple being advanced into the other pre-drilled hole. In some instances, it may be challenging for the surgeon to visualize the locations and trajectories of the pre-drilled holes, for example, due to blood or tissue at or surrounding the surgical site. As a result, it may be difficult for the surgeon to align the arms of the surgical staple with the openings of the pre-drilled holes and ensure that the arms are advanced along the trajectories of the holes, as desired. This may be particularly challenging when the surgical staple is formed of a shape memory material and begins to transform from a deformed configuration to a natural configuration while the surgeon positions the staple relative to the surgical site.

A need therefore exists for improved drill guides for facilitating placement of surgical staples into pre-drilled holes and related methods of using the same, which may overcome one or more of the above-mentioned challenges associated with existing drill guides and their use.

### SUMMARY OF THE DISCLOSURE

The present disclosure provides drill guides for facilitating placement of a surgical staple into pre-drilled holes formed in a patient and related methods of using the same.

In one aspect, a drill guide for facilitating placement of a surgical staple into pre-drilled holes formed in a patient is provided. In one implementation, the drill guide may include a support structure and a plurality of guide members coupled to the support structure. Each of the guide members may have a proximal end and a distal end disposed opposite one another along a longitudinal axis of the guide member. Each of the guide members may include a tubular portion extending from the proximal end toward the distal end of the guide member and a track portion extending from the tubular portion toward the distal end of the guide member. The tubular portion may define a through passage extending along the longitudinal axis of the guide member. The track portion may define an open channel in communication with the through passage and extending along the longitudinal axis of the guide member. The through passage and the open channel may be configured for allowing a drill bit to pass therethrough from the proximal end to the distal end of the guide member to form one of the pre-drilled holes. The open channel may be configured for receiving at least a portion of an arm of the surgical staple therein and guiding the arm of the surgical staple to the distal end of the guide member and into one of the pre-drilled holes.

In some implementations, the guide members may be fixedly coupled to the support structure. In some implementations, the guide members may be removably coupled to the support structure. In some implementations, the longitudinal axes of the guide members may be oriented parallel to one another. In some implementations, the guide members may be spaced apart from one another. In some implementations, the guide members may be spaced apart from one another by a fixed distance. In some implementations, the support structure may include an adjustment mechanism configured for adjusting a spacing between the guide members. In some implementations, the support structure may include a locking mechanism configured for maintaining a spacing between the guide members at a plurality of distances.

In some implementations, the tubular portion may have a first length in the direction of the longitudinal axis of the guide member, the track portion may have a second length in the direction of the longitudinal axis of the guide member, and the first length may be different from the second length. In some implementations, the first length may be less than the second length. In some implementations, the tubular portion may include an inner surface defining the through passage, and the inner surface of the tubular portion may have a circular cross-sectional shape taken perpendicular to the longitudinal axis of the guide member. In some implementations, the track portion may include an inner surface at least partially defining the open channel, and the inner surface of the track portion may have a partial circular cross-sectional shape taken perpendicular to the longitudinal axis of the guide member. In some implementations, a radius of curvature of the inner surface of the tubular portion may be equal to a radius of curvature of the inner surface of the track portion. In some implementations, the tubular portion may include an outer surface having a circular cross-sectional shape taken perpendicular to the longitudinal axis of the guide member. In some implementations, the track portion may include an outer surface having a partial circular cross-sectional shape taken perpendicular to the longitudinal axis of the guide member. In some implementations, a radius of curvature of the outer surface of the tubular portion may be equal to a radius of curvature of the outer surface of the track portion. In some implementations, each of the guide members also may include a plurality of teeth disposed at the distal end of the guide member. In some implementations, each of the teeth may include a sharpened tip. In some implementations, each of the guide members may be formed of a metal. In some implementations, the drill guide also may include a handle coupled to the support structure.

In another aspect, a method of using a drill guide for facilitating placement of a surgical staple into pre-drilled holes formed in a patient is provided. In one embodiment, the method may include positioning the drill guide relative to a surgical site of the patient. The drill guide may include a support structure and a plurality of guide members coupled to the support structure. Each of the guide members may have a proximal end and a distal end disposed opposite one another along a longitudinal axis of the guide member. Each of the guide members may include a tubular portion extending from the proximal end toward the distal end of the guide member and a track portion extending from the tubular portion toward the distal end of the guide member. The tubular portion may define a through passage extending along the longitudinal axis of the guide member. The track portion may define an open channel in communication with the through passage and extending along the longitudinal axis of the guide member. The method also may include advancing a drill bit through the through passage and the open channel of a first guide member of the plurality of guide members and into the patient to form a first pre-drilled hole, and advancing the drill bit through the through passage and the open channel of a second guide member of the plurality of guide members and into the patient to form a second pre-drilled hole. The method also may include positioning at least a portion of a first arm of the surgical staple within the open channel of the first guide member and at least a portion of a second arm of the surgical staple within the open channel of the second guide member. The method also may include advancing the first arm of the surgical staple along the open channel of the first guide member and into the first pre-drilled hole and the second arm of the surgical staple along the open channel of the second guide member and into the second pre-drilled hole.

In some implementations, the guide members may be fixedly coupled to the support structure. In some implementations, the guide members may be removably coupled to the support structure. In some implementations, the longitudinal axes of the guide members may be oriented parallel to one another. In some implementations, the guide members may be spaced apart from one another. In some implementations, the guide members may be spaced apart from one another by a fixed distance. In some implementations, the support structure may include an adjustment mechanism configured for adjusting a spacing between the guide members. In some implementations, the support structure may include a locking mechanism configured for maintaining a spacing between the guide members at a plurality of distances.

In some implementations, the tubular portion may have a first length in the direction of the longitudinal axis of the guide member, the track portion may have a second length in the direction of the longitudinal axis of the guide member, and the first length may be different from the second length. In some implementations, the first length may be less than the second length. In some implementations, the tubular portion may include an inner surface defining the through passage, and the inner surface of the tubular portion may have a circular cross-sectional shape taken perpendicular to the longitudinal axis of the guide member. In some implementations, the track portion may include an inner surface at least partially defining the open channel, and the inner surface of the track portion may have a partial circular cross-sectional shape taken perpendicular to the longitudinal axis of the guide member. In some implementations, a radius of curvature of the inner surface of the tubular portion may be equal to a radius of curvature of the inner surface of the track portion. In some implementations, the tubular portion may include an outer surface having a circular cross-sectional shape taken perpendicular to the longitudinal axis of the guide member. In some implementations, the track portion may include an outer surface having a partial circular cross-sectional shape taken perpendicular to the longitudinal axis of the guide member. In some implementations, a radius of curvature of the outer surface of the tubular portion may be equal to a radius of curvature of the outer surface of the track portion. In some implementations, each of the guide members also may include a plurality of teeth disposed at the distal end of the guide member, and positioning the drill guide relative to the surgical site may include engaging the patient with the teeth. In some implementations, each of the teeth may include a sharpened tip. In some implementations, each of the guide members may be formed of a metal. In some implementations, the drill guide also may include a handle coupled to the support structure.

In some implementations, the surgical staple may be formed of a shape memory material. In some implementations, the surgical staple may be configured to be deformed from a natural configuration to a deformed configuration, and the surgical staple may be in the deformed configuration when the at least a portion of the first arm of the surgical staple is positioned within the open channel of the first guide member and the at least a portion of the second arm of the surgical staple is positioned within the open channel of the second guide member. In some implementations, the surgical staple may be allowed to transition from the deformed configuration toward the natural configuration after the first arm of the surgical staple is advanced along the open channel of the first guide member and into the first pre-drilled hole and the second arm of the surgical staple is advanced along the open channel of the second guide member and into the second pre-drilled hole. In some implementations, the first pre-drilled hole may be formed in a first bone structure of the patient, and the second pre-drilled hole may be formed in a second bone structure of the patient. In some implementations, the first pre-drilled hole and the second pre-drilled hole may be spaced apart from one another across an osteotomy site of the patient.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A is a top view of an example guide member as may be used as a part of a drill guide in accordance with implementations of the disclosure, showing a tubular portion, a track portion, and teeth of the guide member.

FIG. 1B is a cross-sectional side view of the guide member of FIG. 1A, taken along line B-B of FIG. 1A, showing a through passage of the tubular portion and an open channel of the track portion.

FIG. 1C is an end view of the guide member of FIG. 1A.

FIGS. 2A and 2B are perspective views of two of the guide members of FIG. 1A, showing arms of an example surgical staple being advanced along the open channels of the track portions of the guide members.

FIG. 3 is a perspective view of an example drill guide in accordance with implementations of the disclosure, showing the drill guide including four of the guide members of FIG. 1A and a support structure.

FIG. 4A is a perspective view of an example drill guide in accordance with implementations of the disclosure, showing the drill guide including two of the guide members of FIG. 1A, a support structure including an adjustment mechanism and a locking mechanism, and a handle.

FIG. 4B is a perspective view of the drill guide of FIG. 4A.

FIGS. 4C and 4D are perspective views of the drill guide of FIG. 4A, showing arms of an example surgical staple being advanced along the open channels of the track portions of the guide members using an example staple insertion tool.

FIG. 4E is a perspective view of the drill guide of FIG. 4A, showing arms of an example surgical staple being advanced along the open channels of the track portions of the guide members using an example staple insertion tool.

FIG. 4F is a detailed perspective view of portions of the drill guide, the staple insertion tool, and the surgical staple of FIG. 4E.

FIG. 4G is a front view of the drill guide, the staple insertion tool, and the surgical staple of FIG. 4E.

FIG. 4H is a detailed front view of portions of the drill guide, the staple insertion tool, and the surgical staple of FIG. 4G.

FIG. 5A is a perspective view of an example drill guide in accordance with implementations of the disclosure, showing the drill guide including two of the guide members of FIG. 1A, a support structure including an adjustment mechanism and a locking mechanism, and a handle.

FIG. 5B is a perspective view of the drill guide of FIG. 5A.

FIG. 5C is a detailed perspective view of portions of the drill guide of FIG. 5A.

FIG. 5D is a detailed perspective view of portions of the drill guide of FIG. 5A.

The detailed description is set forth with reference to the accompanying drawings. The drawings are provided for purposes of illustration only and merely depict example implementations of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various implementations may utilize elements or components other than those illustrated in the drawings, and some elements and/or components may not be present in various implementations. The use of singular terminology to describe a component or element may, depending on the context, encompass a plural number of such components or elements and vice versa.

### DETAILED DESCRIPTION OF THE DISCLOSURE

In the following description, specific details are set forth describing some implementations consistent with the present disclosure. Numerous specific details are set forth in order to provide a thorough understanding of the implementations. It will be apparent, however, to one skilled in the art that some implementations may be practiced without some or all of these specific details. The specific implementations disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other implementations unless specifically described otherwise or if the one or more features would make an implementation non-functional. In some instances, well known methods, procedures, and components have not been described in detail so as not to unnecessarily obscure aspects of the implementations.

### Overview

Implementations of drill guides for facilitating placement of a surgical staple into pre-drilled holes formed in a patient and related methods of using the same are provided. As described herein, the drill guides may be used to guide a drill bit in forming drilled holes in a patient and then to facilitate placement of a surgical staple into the pre-drilled holes. In some implementations, the pre-drilled holes may be formed in respective bone structures of the patient. In some implementations, the pre-drilled holes may be spaced apart from one another across an osteotomy site of the patient. In some implementations, the surgical staple may be formed of a shape memory material and configured to be deformed from a natural configuration to a deformed configuration. In some implementations, the drill guide may maintain the surgical staple in the deformed configuration while the drill guide guides the surgical staple toward the pre-drilled holes, and then the surgical staple may be allowed to transition from the deformed configuration toward the natural configuration when the arms of the surgical staple are advanced out of the drill guide and into the pre-drilled holes.

As described herein, a drill guide for facilitating placement of a surgical staple into pre-drilled holes formed in a patient generally may include a support structure and a plurality of guide members coupled to the support structure. Each of the guide members may have a proximal end and a distal end disposed opposite one another along a longitudinal axis of the guide member. Each of the guide members may include a tubular portion extending from the proximal end toward the distal end of the guide member and a track portion extending from the tubular portion toward the distal end of the guide member. The tubular portion may define a through passage extending along the longitudinal axis of the guide member. The track portion may define an open channel in communication with the through passage and extending along the longitudinal axis of the guide member. The through passage and the open channel may be configured for allowing a drill bit to pass therethrough from the proximal end to the distal end of the guide member to form one of the pre-drilled holes. The open channel may be configured for receiving at least a portion of an arm of the surgical staple therein and guiding the arm of the surgical staple to the distal end of the guide member and into one of the pre-drilled holes.

As discussed above, certain existing drill guides may be used solely to guide drilling of holes at a surgical site of a patient and then must be removed from the surgical site to allow for placement of a surgical staple into the pre-drilled holes. In other words, such drill guides may lack a means for facilitating placement of the surgical staple into the pre-drilled holes. In some instances, it may be challenging for the surgeon to visualize the locations and trajectories of the pre-drilled holes, for example, due to blood or tissue at or surrounding the surgical site. As a result, it may be difficult for the surgeon to align the arms of the surgical staple with the openings of the pre-drilled holes and ensure that the arms are advanced along the trajectories of the holes, as desired. This may be particularly challenging when the surgical staple is formed of a shape memory material and begins to transform from a deformed configuration to a natural configuration while the surgeon positions the staple relative to the surgical site. The drill guides and related methods provided herein advantageously may overcome these challenges. As described, the present drill guides need not be removed from the surgical site after being used to guide drilling of holes formed in the patient. Rather, the drill guides are intended to remain in place and configured to guide the arms of a surgical staple into the pre-drilled holes. In this manner, the surgeon may be able to advance the surgical staple efficiently and confidently into the pre-drilled holes, without having to directly visualize the locations and trajectories of the pre-drilled holes.

Still other benefits and advantages of the drill guides and methods provided herein over existing drill guides and techniques will be appreciated by those of ordinary skill in the art from the following description and the appended drawings.

### Example Drill Guides and Methods

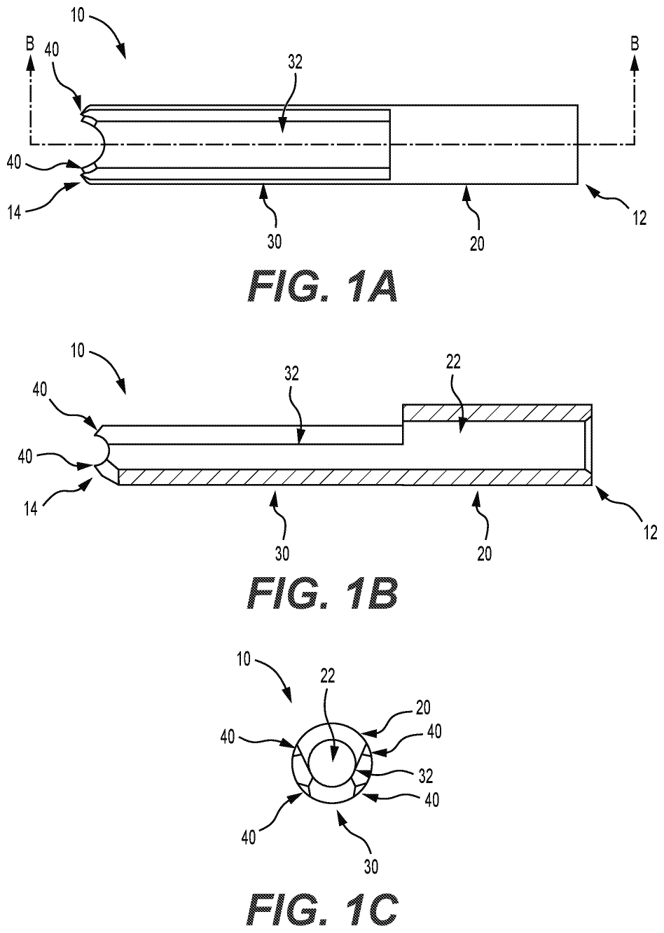

Referring now to FIGS. 1A-1C, an example guide member 10 is depicted. As described below, multiple guide members 10 may be used as part of a drill guide in accordance with implementations of the present disclosure. Various configurations of the guide member 10 may be used in different implementations, for example to accommodate different types or sizes of drill bits as well as different types or sizes of surgical staples. The guide member 10 may have an elongate shape, with a proximal end 12 and a distal end 14 disposed opposite one another along a longitudinal axis of the guide member 10. As shown, the guide member 10 may include a tubular portion 20 and a track portion 30. The tubular portion 20 may extend from the proximal end 12 toward the distal end 14 of the guide member 10. The track portion 30 may extend from the tubular portion 20 toward the distal end 14 of the guide member 10. In some implementations, as shown, the guide member 10 also may include a plurality of teeth 40 disposed at the distal end 14 of the guide member 10. The guide member 10 may be formed as a rigid member. In various implementations, the guide member 10 may be formed of a metal, a polymer, a ceramic, a composite, or another suitable material or combinations thereof.

As shown, the tubular portion 20 may define a through passage 22 extending along the longitudinal axis of the guide member 10, and the track portion 30 may define an open channel 32 in communication with the through passage 22 and extending along the longitudinal axis of the guide member 10. The through passage 22 and the open channel 32 may be configured for allowing a drill bit to pass therethrough from the proximal end 12 to the distal end 14 of the guide member 10. As described below, a drill bit may be advanced through and guided by the through passage 22 and the open channel 32 when using the guide member 10 to drill a hole in a patient. The open channel 32 also may be configured for receiving at least a portion of an arm of a surgical staple therein and guiding the arm of the surgical staple to the distal end 14 of the guide member 10 and into the pre-drilled hole. The teeth 40 may be configured to facilitate positioning of the guide member 10 relative to the patient and to maintain a desired position and orientation of the guide member 10 while the guide member 10 is used to drill the hole in the patient and guide the arm of the surgical staple into the pre-drilled hole. In some examples, as shown, each of the teeth 40 may include a sharpened tip for securely engaging the patient at the surgical site. Various shapes of the teeth 40 may be used in different implementations.

According to the illustrated example, the tubular portion 20 and the track portion 30 may have different lengths. As shown, the tubular portion 20 may have a first length in the direction of the longitudinal axis of the guide member 10, and the track portion 30 may have a second length in the direction of the longitudinal axis of the guide member 10, with the first length being less than the second length. Other relationships of the lengths of the tubular portion 20 and the track portion 30 may be used in other implementations. As shown, the tubular portion 20 may include an inner surface that defines the through passage 22 and has a circular cross-sectional shape taken perpendicular to the longitudinal axis of the guide member 10. As shown, the track portion 30 may include an inner surface that at least partially defines the open channel 32 and has a partial circular cross-sectional shape taken perpendicular to the longitudinal axis of the guide member 10. In some implementations, as shown, a radius of curvature of the inner surface of the tubular portion 20 may be equal to a radius of curvature of the inner surface of the track portion 30. Other cross-sectional shapes of the inner surface of the tubular portion 20 and the inner surface of the track portion 30 and relationships therebetween may be used in other implementations. As shown, the tubular portion 20 may include an outer surface that has a circular cross-sectional shape taken perpendicular to the longitudinal axis of the guide member 10. As shown, the track portion 30 may include an outer surface that has a partial circular cross-sectional shape taken perpendicular to the longitudinal axis of the guide member 10. In some implementations, as shown, a radius of curvature of the outer surface of the tubular portion 20 may be equal to a radius of curvature of the outer surface of the track portion 30. Other cross-sectional shapes of the outer surface of the tubular portion 20 and the outer surface of the track portion 30 and relationships therebetween may be used in other implementations.

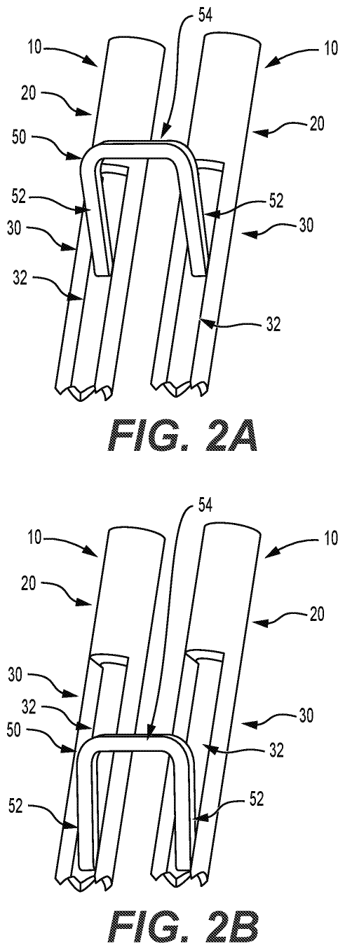

FIGS. 2A and 2B illustrate two of the guide members 10 being used to guide a surgical staple 50. As shown, the open channels 32 have similar orientation, shape, and length. The surgical staple 50 may include a pair of arms 52 connected to one another by a cross-member 54. As shown, end portions of the respective arms 52 may be positioned within the open channels 32 of the respective guide members 10 and then advanced along the open channels 32 toward the distal ends 14 of the guide members 10.

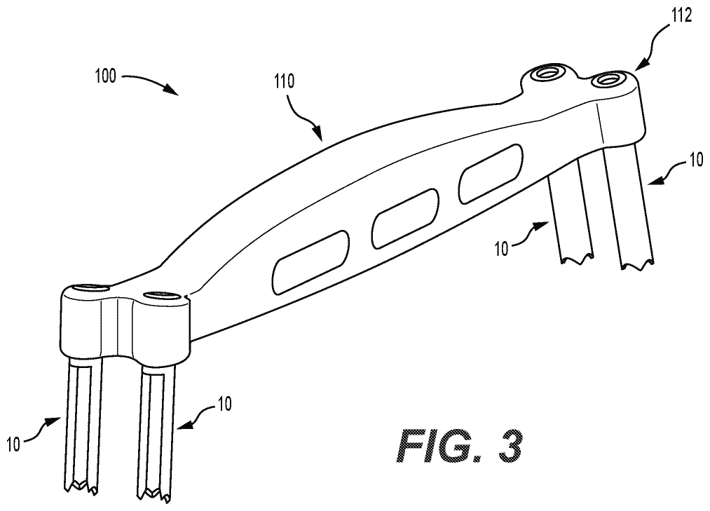

FIG. 3 illustrates an example drill guide 100 in accordance with implementations of the present disclosure. As shown, the drill guide 100 may include a support structure 110 and four guide members 10 configured in the manner described above. In some implementations, as shown, the support structure 110 may be formed as an elongate member having a first end 112 and a second end 114 disposed opposite one another along a longitudinal axis of the support structure 110. In some implementations, as shown, a first pair of the guide members 10 may be coupled to the support structure 110 near the first end 112 thereof and a second pair of the guide members 10 may be coupled to the support structure 110 near the second end 114 thereof. Other arrangements of the guide members 10 relative to the support structure 110 may be used in other implementations. The support structure 110 may be formed as a rigid member. In various implementations, the support structure 110 may be formed of a metal, a polymer, a ceramic, a composite, or another suitable material or combinations thereof.

In some implementations, the guide members 10 may be fixedly coupled to the support structure 110. In some implementations, the guide members 10 may be removably coupled to the support structure 110, for example, to allow for different sizes or shapes of the guide members 10 to be used with the support structure 110. In some implementations, as shown, for each pair of the guide members 10, the longitudinal axes of the guide members 10 may be oriented parallel to one another. Meanwhile, according to the illustrated example, the longitudinal axes of the guide members 10 of the first pair may be oriented at an acute angle relative to the longitudinal axes of the guide members 10 of the second pair. In some implementations, as shown, for each pair of the guide members 10, the guide members 10 may be spaced apart from one another. According to the illustrated example, for each pair of the guide members 10, the guide members 10 may be spaced apart from one another by a fixed distance. In other implementations, the support structure 110 may include an adjustment mechanism configured for adjusting a spacing between the guide members 10 of each pair. In such implementations, the support structure 110 also may include a locking mechanism configured for maintaining a spacing between the guide members 10 of each pair at a plurality of different distances. Various arrangements of the support structure 110 and the guide members 10 (and corresponding open channels 32) may be used in different embodiments.

During use, the drill guide 100 first may be positioned relative to a surgical site of a patient, as desired by the surgeon. Then, with respect to each of the guide members 10, a drill bit may be advanced through the through passage 22 and the open channel 32 of the guide member 10 and into the patient to form a hole. Next, with respect to each pair of the guide members 10, a surgical staple 50 may be positioned with respect to the guide members 10 in the manner described above with respect to FIGS. 2A and 2B. In particular, an end portion of one of the arms 52 of the surgical staple 50 may be positioned within the open channel 32 of one of the guide members 10, and an end portion of the other arm 52 of the surgical staple 50 may be positioned within the open channel 32 of the other guide member 10. Then, the arms 52 of the surgical staple 50 may be advanced along the open channels 32 of the respective guide members 10 and into the respective pre-drilled holes. After advancing the arms 52 of the surgical staple 50 into the pre-drilled holes, the drill guide 100 may be removed from the surgical site.

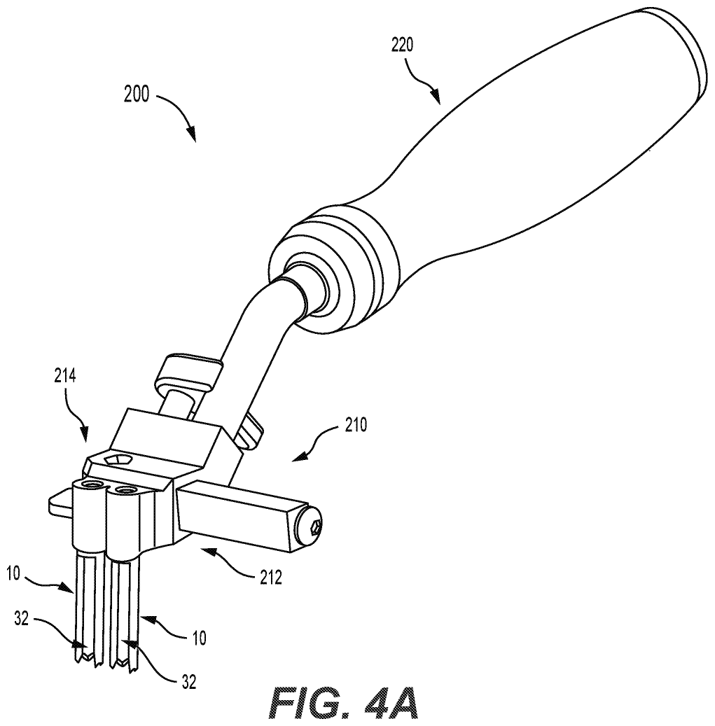

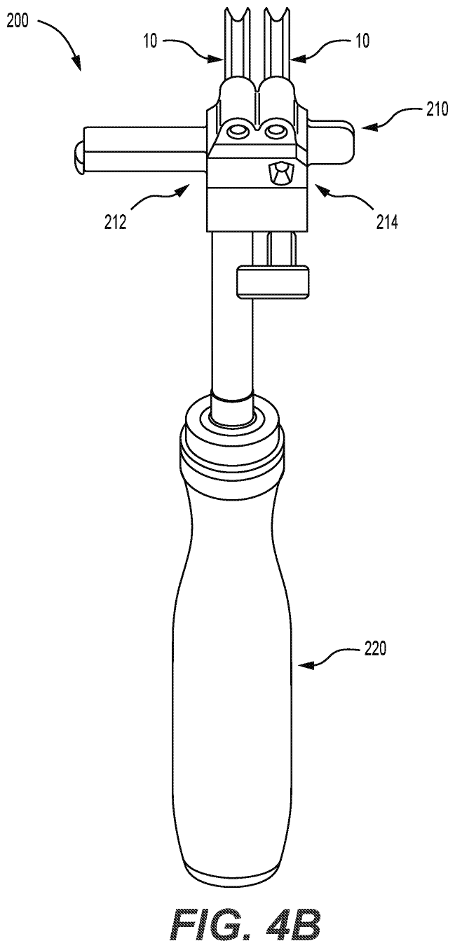

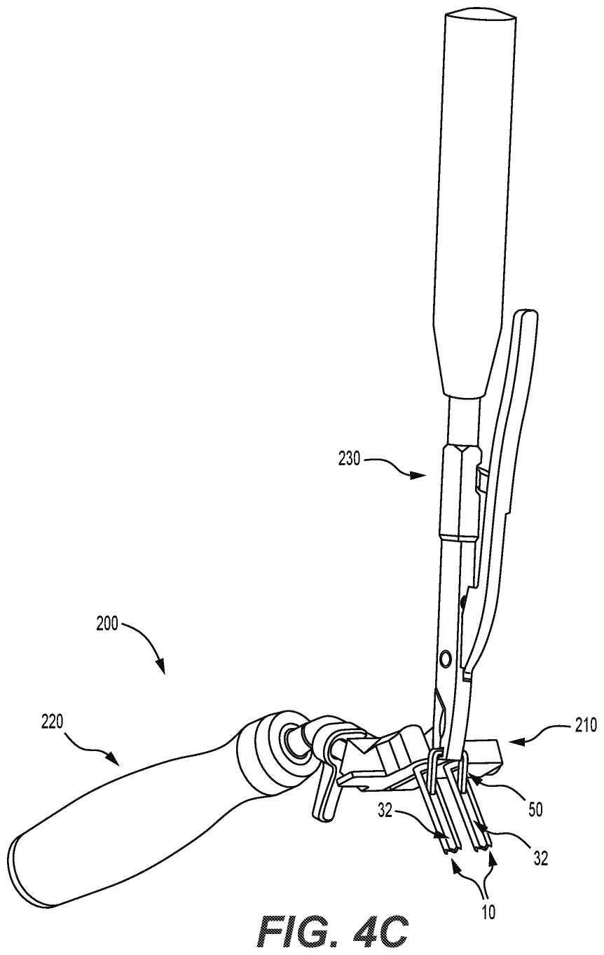

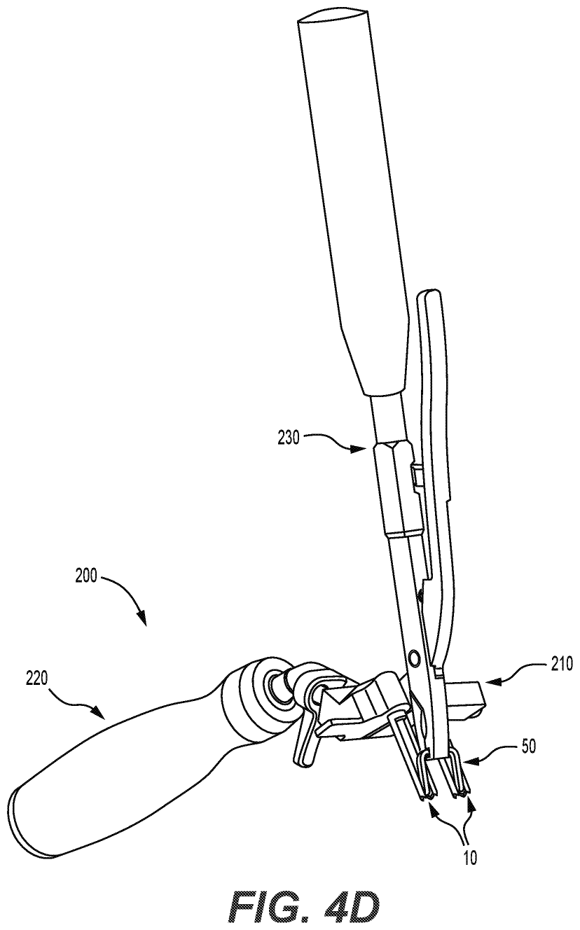

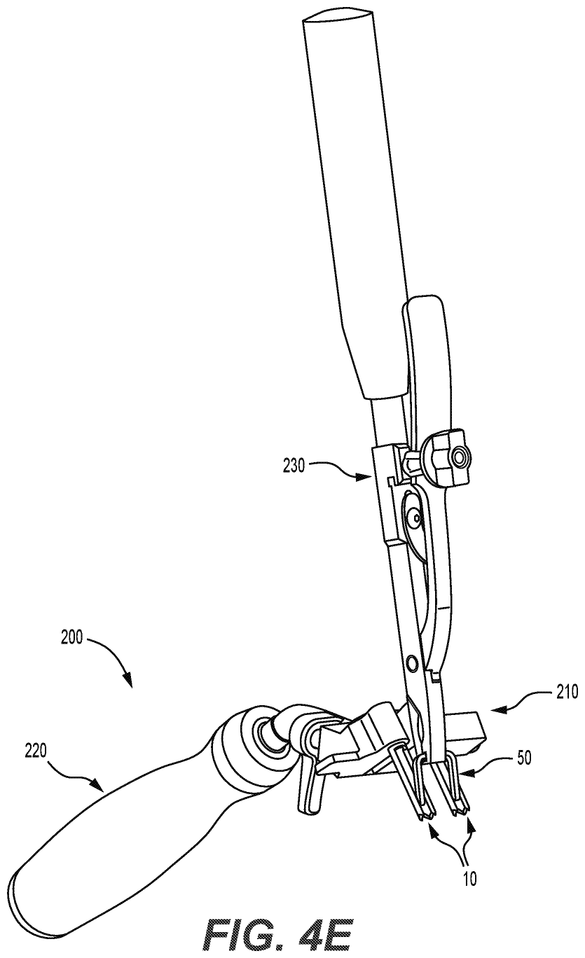

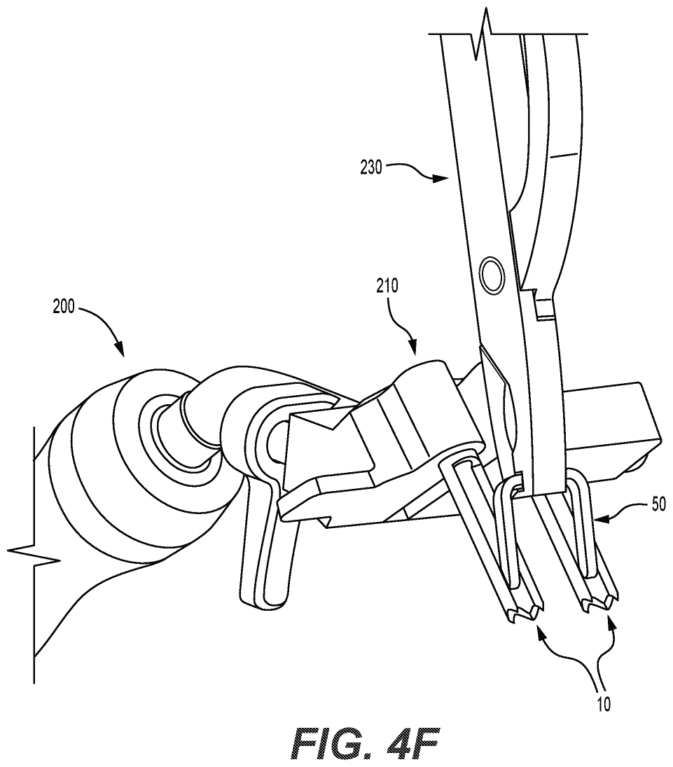

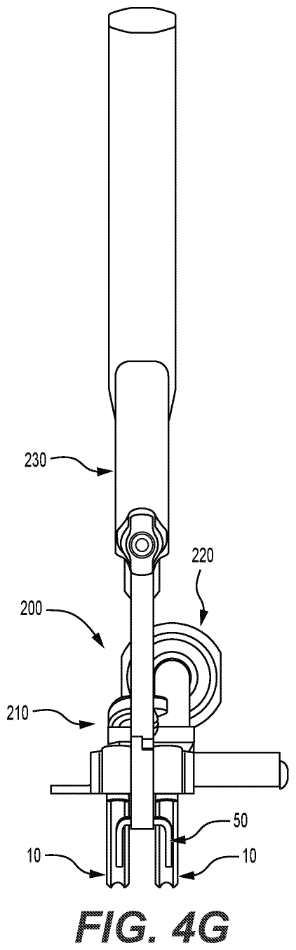

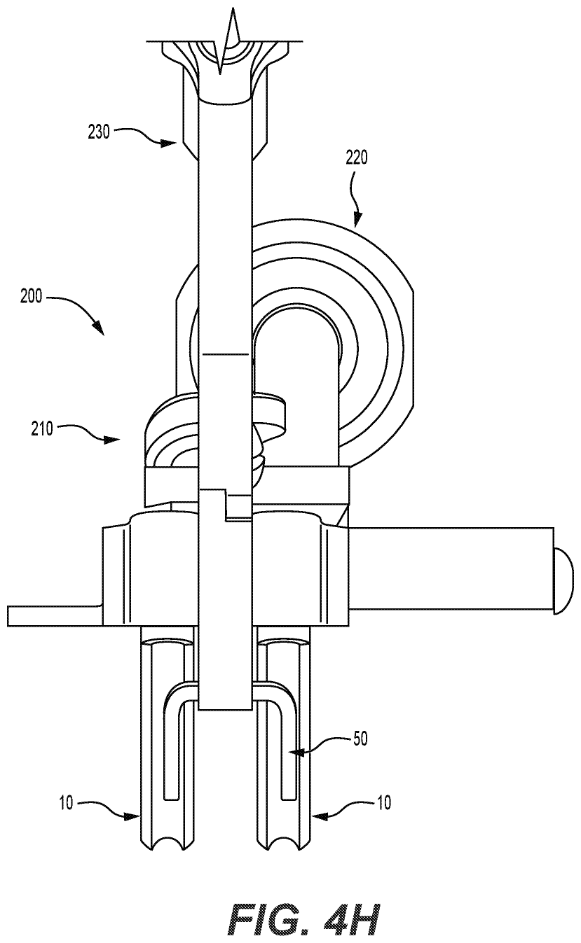

FIGS. 4A-4H illustrate another example drill guide 200 in accordance with implementations of the present disclosure. As shown, the drill guide 200 may include a support structure 210, a handle 220, and two guide members 10 configured in the manner described above. In some implementations, as shown, the support structure 210 may be formed as an assembly of components for supporting the guide members 10 and also for adjusting and maintaining a spacing between the guide members 10. Various arrangements of the guide members 10 relative to the support structure 210 and the handle 220 may be used in different implementations. In various implementations, the components of the support structure 210 may be formed of a metal, a polymer, a ceramic, a composite, or another suitable material or combinations thereof.

In some implementations, the guide members 10 may be fixedly coupled to the support structure 210, 310. In some implementations, the guide members 10 may be removably coupled to the support structure 210, for example, to allow for different sizes or shapes of the guide members 10 to be used with the support structure 210. In some implementations, as shown, the longitudinal axes of the guide members 10 may be oriented parallel to one another.

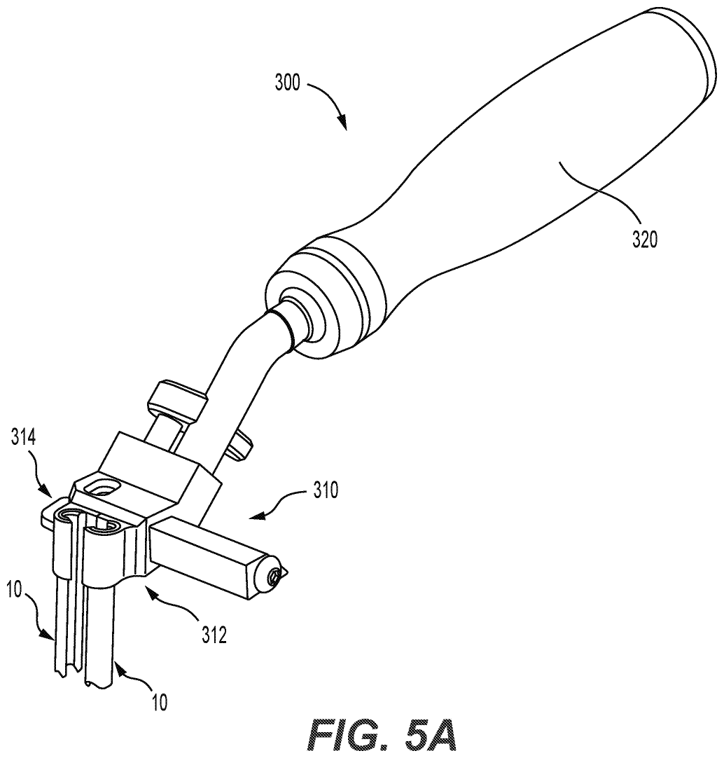

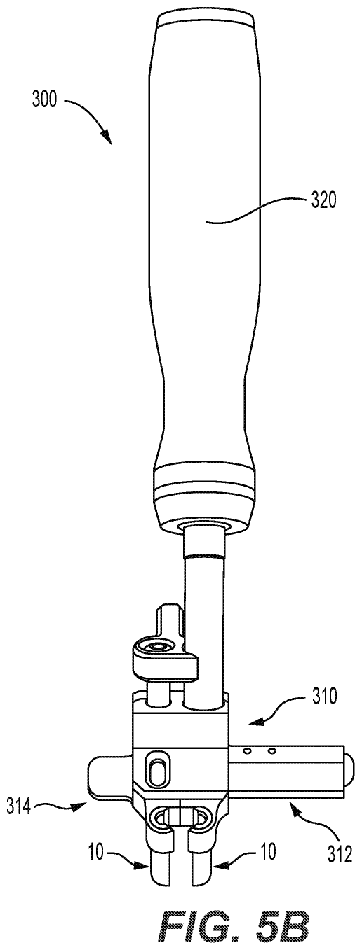

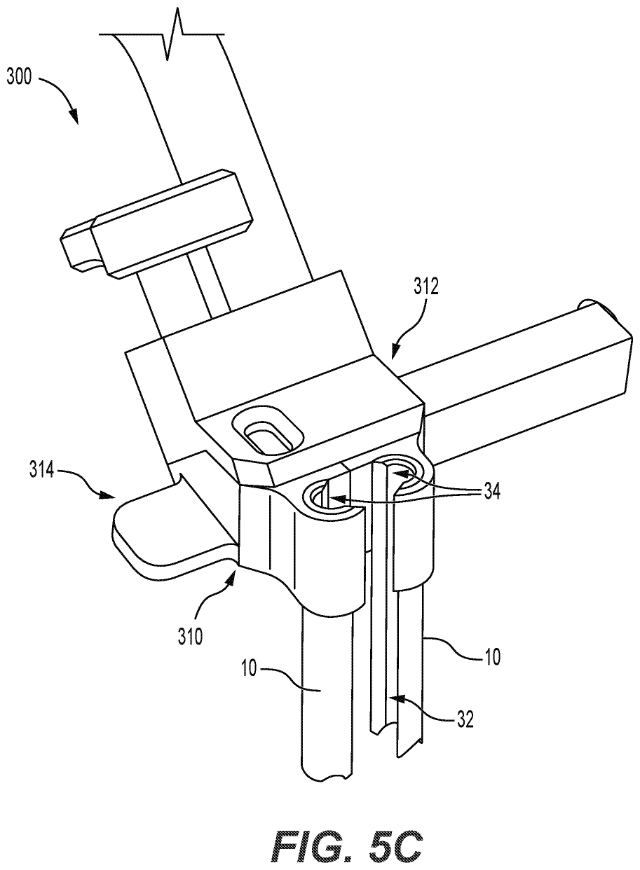

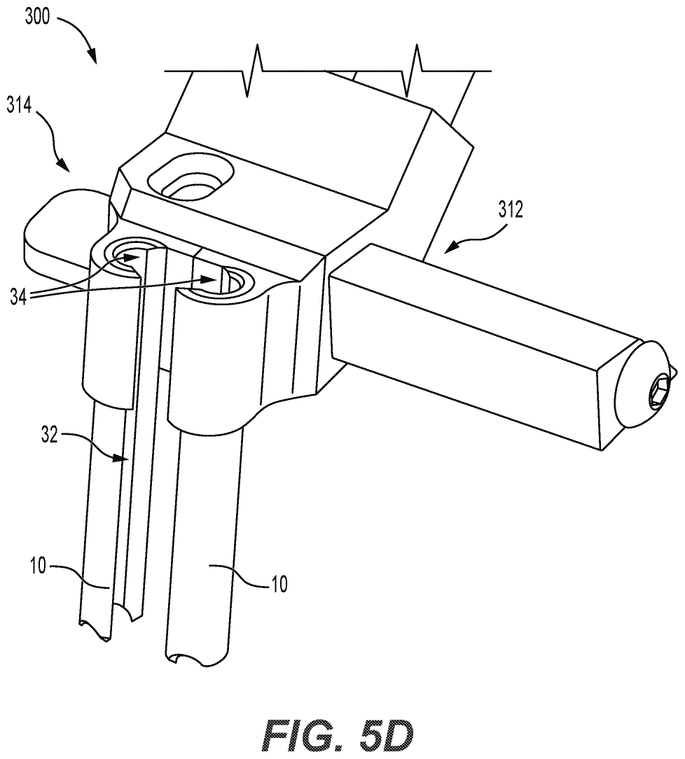

In some implementations, as illustrated in FIGS. 4A-4H, the guide members 10 include open channels 32 having similar orientation, shape, and/or length. In further implementations, the guide members 10 include opening channels 32 having different/varying orientation, shape, and/or length. For example, FIGS. 5A-5D illustrate another example drill guide 300 in accordance with a further implementation of the present disclosure having opening channels 32 oriented to face one another. The example drill guide 300 is similar to the drill guide 200 of FIGS. 4A-4H, and like reference numbers are used to identify like features, the difference between the drill guide 200 and drill guide 300 is described in more detail below. As illustrated in FIGS. 5A-5D, the open channels 32 of the drill guide 300 have similar size and shape, and the open portion of the open channel 32 of each of the guide members is oriented to face the opposing open portion of the opposite guide channel 32. As illustrated in FIGS. 5A-5D, the drill guide 300 includes a support structure 310, a handle 320, and two guide members 10 configured in the manner described above. In some implementations, as shown, the support structure 310 may be formed as an assembly of components for supporting the guide members 10 and also for adjusting and maintaining a spacing between the guide members 10. As illustrated in FIGS. FIGS. 5A-5D, the support structure 310 includes an opening for providing access to and between the open channels 32 of each of the guide members 10 retained within the support structure 310. Various arrangements of the guide members 10 relative to the support structure 310 and the handle 320 may be used in different implementations.

In some implementations, as shown in FIGS. 4A-5D, the guide members 10 may be spaced apart from one another. According to the illustrated example, the support structure 210, 310 may include an adjustment mechanism 212, 312 configured for adjusting a spacing between the guide members 10. In some implementations, as shown, the support structure 210, 310 also may include a locking mechanism 214, 314 configured for maintaining a spacing between the guide members 10 at a plurality of different distances. In this manner, the adjustment mechanism 212, 312 may be used to allow the surgeon to adjust the spacing between the guide members 10 to a desired distance, and the locking mechanism 214, 314 may be used to lock the guide members 10 at the desired distance. Various arrangements of the support structure 210, 310 and the guide members 10 may be used in different implementations.

During use, the drill guide 200, 300 first may be positioned relative to a surgical site of a patient, as desired by the surgeon. In some implementations, the spacing between the guide members 10 may be adjusted and then locked using the adjustment mechanism 212, 312 and the locking mechanism 214, 314, respectively. Then, with respect to each of the guide members 10, a drill bit may be advanced through the through passage 22 and the open channel 32 of the guide member 10 and into the patient to form a hole. In reference to the drill guide 200 provided in FIGS. 4A-4H, next, a surgical staple 50 may be positioned with respect to the guide members 10, as shown in FIG. 4C. In particular, an end portion of one of the arms 52 of the surgical staple 50 may be positioned within the open channel 32 of one of the guide members 10, and an end portion of the other arm 52 of the surgical staple 50 may be positioned within the open channel 32 of the other guide member 10. Then, the arms 52 of the surgical staple 50 may be advanced along the open channels 32 of the respective guide members 10, as shown in FIGS. 4D-4H, and into the respective pre-drilled holes. In some implementations, as shown, a staple insertion tool 230 may be used to position the surgical staple 50 relative to the guide members 10 and to advance the arms 52 of the surgical staple 50 along the open channels 32 into the pre-drilled holes. After advancing the arms 52 of the surgical staple 50 into the pre-drilled holes, the surgical staple 50 may be released from the staple insertion tool 230, and the drill guide 200 may be removed from the surgical site.

In reference to the drill guide 300 provided in FIGS. 5A-5D, the surgical staple 50 may be positioned at the top opening 34 of the open channel 32. In particular, the end portions of each of the arms 52 of the surgical staple 50 may be positioned in the portion of the top opening 34 corresponding to the desired open channel 32. The arms 52 of the surgical stable 50 may be advanced along the open channels 32 of the respective guide members 10. In some implementations, the arms 52 of the surgical staple 50 are retained within the through passage 22 of the corresponding guide members 10 and into the respective pre-drilled holes. In some implementations, a staple insertion tool 230 similar to the one illustrated in FIGS. 4C-4H may be used to position the surgical staple 50 relative to the guide members 10 and to advance the arms 52 of the surgical staple 50 along the open channels 32 into the pre-drilled holes. After advancing the arms 52 of the surgical staple 50 into the pre-drilled holes, the surgical staple 50 may be released from the staple insertion tool 230, and the drill guide 200 may be removed from the surgical site.

Although specific implementations of the disclosure have been described, one of ordinary skill in the art will recognize that numerous other modifications and alternative implementations are within the scope of the disclosure. For example, while various illustrative implementations and structures have been described in accordance with examples of the disclosure, one of ordinary skill in the art will appreciate that numerous other modifications to the illustrative implementations and structures described herein are also within the scope of this disclosure.

Although implementations have been described in language specific to structural features and/or methodological acts, it is to be understood that the disclosure is not necessarily limited to the specific features or acts described. Rather, the specific features and acts are disclosed as illustrative forms of implementing the implementations. Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or steps. Thus, such conditional language is not generally intended to imply that features, elements, and/or steps are in any way required for one or more implementations.

1. A drill guide for facilitating placement of a surgical staple into pre-drilled holes formed in a patient, the drill guide comprising:
a support structure; and
a plurality of guide members coupled to the support structure, each of the guide members having a proximal end and a distal end disposed opposite one another along a longitudinal axis of the guide member, and each of the guide members comprising:
a tubular portion extending from the proximal end toward the distal end of the guide member, the tubular portion defining a through passage extending along the longitudinal axis of the guide member; and
a track portion extending from the tubular portion toward the distal end of the guide member, the track portion defining an open channel in communication with the through passage and extending along the longitudinal axis of the guide member;
wherein the through passage and the open channel are configured for allowing a drill bit to pass therethrough from the proximal end to the distal end of the guide member to form one of the pre-drilled holes; and
wherein the open channel is configured for receiving at least a portion of an arm of the surgical staple therein and guiding the arm of the surgical staple to the distal end of the guide member and into one of the pre-drilled holes.

2. The drill guide of claim 1, wherein the guide members are fixedly coupled to the support structure.

3. The drill guide of claim 1, wherein the guide members are removably coupled to the support structure.

4. (canceled)

5. (canceled)

6. The drill guide of claim 1, wherein the guide members are spaced apart from one another by a fixed distance.

7. The drill guide of claim 1, wherein the support structure comprises an adjustment mechanism configured for adjusting a spacing between the guide members.

8. The drill guide of claim 7, wherein the support structure comprises a locking mechanism configured for maintaining a spacing between the guide members at a plurality of distances.

9. (canceled)

10. (canceled)

11. The drill guide of claim 1, wherein the tubular portion comprises an inner surface defining the through passage, and wherein the inner surface of the tubular portion has a circular cross-sectional shape taken perpendicular to the longitudinal axis of the guide member.

12. The drill guide of claim 11, wherein the track portion comprises an inner surface at least partially defining the open channel, and wherein the inner surface of the track portion has a partial circular cross-sectional shape taken perpendicular to the longitudinal axis of the guide member.

13. (canceled)

14. (canceled)

15. (canceled)

16. (canceled)

17. The drill guide of claim 1, wherein each of the guide members further comprises a plurality of teeth disposed at the distal end of the guide member,
wherein each of the teeth comprises a sharpened tip.

18. (canceled)

19. A method of using a drill guide for facilitating placement of a surgical staple into pre-drilled holes formed in a patient, the method comprising:
positioning the drill guide relative to a surgical site of the patient, the drill guide comprising:
a support structure; and
a plurality of guide members coupled to the support structure, each of the guide members having a proximal end and a distal end disposed opposite one another along a longitudinal axis of the guide member, and each of the guide members comprising:
a tubular portion extending from the proximal end toward the distal end of the guide member, the tubular portion defining a through passage extending along the longitudinal axis of the guide member; and
a track portion extending from the tubular portion toward the distal end of the guide member, the track portion defining an open channel in communication with the through passage and extending along the longitudinal axis of the guide member;
advancing a drill bit through the through passage and the open channel of a first guide member of the plurality of guide members and into the patient to form a first pre-drilled hole;
advancing the drill bit through the through passage and the open channel of a second guide member of the plurality of guide members and into the patient to form a second pre-drilled hole;
positioning at least a portion of a first arm of the surgical staple within the open channel of the first guide member and at least a portion of a second arm of the surgical staple within the open channel of the second guide member; and
advancing the first arm of the surgical staple along the open channel of the first guide member and into the first pre-drilled hole and the second arm of the surgical staple along the open channel of the second guide member and into the second pre-drilled hole.

20. (canceled)

21. The method of claim 19, wherein the guide members are removably coupled to the support structure.

22. (canceled)

23. (canceled)

24. The method of claim 19, wherein the guide members are spaced apart from one another by a fixed distance.

25. The method of claim 19, wherein the support structure comprises an adjustment mechanism configured for adjusting a spacing between the guide members.

26. The method of claim 25, wherein the support structure comprises a locking mechanism configured for maintaining a spacing between the guide members at a plurality of distances.

27. (canceled)

28. (canceled)

29. The method of claim 19, wherein the tubular portion comprises an inner surface defining the through passage, and wherein the inner surface of the tubular portion has a circular cross-sectional shape taken perpendicular to the longitudinal axis of the guide member.

30. (canceled)

31. (canceled)

32. (canceled)

33. (canceled)

34. (canceled)

35. The method of claim 19, wherein each of the guide members further comprises a plurality of teeth disposed at the distal end of the guide member,
wherein each of the teeth comprises a sharpened tip, and
wherein positioning the drill guide relative to the surgical site comprises engaging the patient with the teeth.

36. (canceled)

37. The method of claim 19, wherein the surgical staple is formed of a shape memory material,
wherein the surgical staple is configured to be deformed from a natural configuration to a deformed configuration, and wherein the surgical staple is in the deformed configuration when the at least a portion of the first arm of the surgical staple is positioned within the open channel of the first guide member and the at least a portion of the second arm of the surgical staple is positioned within the open channel of the second guide member.

38. The method of claim 37, wherein the surgical staple is allowed to transition from the deformed configuration toward the natural configuration after the first arm of the surgical staple is advanced along the open channel of the first guide member and into the first pre-drilled hole and the second arm of the surgical staple is advanced along the open channel of the second guide member and into the second pre-drilled hole.

39. The method of claim 19, wherein the first pre-drilled hole is formed in a first bone structure of the patient, and wherein the second pre-drilled hole is formed in a second bone structure of the patient.

40. The method of claim 19, wherein the first pre-drilled hole and the second pre-drilled hole are spaced apart from one another across an osteotomy site of the patient.
